(19) United States
(12) Patent Application Publication
(10) Pub. No.: US 2005/0003027 A1
(43) Pub. Date: Jan. 6, 2005

(54) Chemical composition and method to bind fat, enhance metabolization, and aid digestion

(76) Inventors: Jose A. Diaz, Coconut Grove, FL (US); Eduardo M. Naranjo, Coconut Grove, FL (US)

Correspondence Address: MALLOY & MALLOY, P. A., 2800 S. W. Third Avenue, Historic Coral Way, Miami, FL, 33129, US

(21) Appl. No.: 10/841,259

(22) Filed: May 7, 2004

Related U.S. Application Data
(60) Provisional application No. 60/469,121, filed on May 9, 2003.

(51) Int. Cl. A61K 35/78

(52) U.S. Cl. 424/738; 424/769; 424/757; 514/55

(57) ABSTRACT

A chemical composition and a method to inhibit fat absorption, enhance metabolization of carbohydrates, and maintain normal functioning of the digestive processes in a person's body. Specifically, the chemical composition and method are utilized to bind fat thereby limiting absorption by the body, enhance the metabolization of carbohydrates, such as sugars, to limit storage in the body, and maintain the digestive processes to facilitate elimination of unwanted fats and carbohydrates from the body. One formulation of the chemical composition includes a mixture of a fat binding fraction, which comprises between approximately 22% and 27% by weight, a metabolic enhancer fraction comprising between approximately 43% and 53% by weight, and a digestive aid fraction comprising between approximately 25% and 31% by weight of the total chemical composition. A mixture of vitamins, minerals, and other beneficial compounds are included in the chemical composition and utilized by the method.

### CLAIM OF PRIORITY

The present application is based on and a claim to priority is made under 35 U.S.C. Section 119(e) to provisional patent application currently pending in the U.S. Patent and Trademark Office having Ser. No. 60/469,121 and a filing date of May 9, 2003.

### BACKGROUND OF THE INVENTION

1. Field of the Invention

The present invention relates to a chemical composition structured to bind to fat molecules and reduce their absorption by the human body, as well as to enhance the body's metabolization of carbohydrates, such as sugars, so as to reduce blood glucose levels, and thereby, minimize subsequent storage of glucose in the body. The inventive composition further aids with maintaining normal functioning of the digestive processes to facilitate elimination of unwanted fats and carbohydrates from the body. The present invention also comprises a method involving the preparation and ingestion of the chemical composition in recommended dosages.

2. Description of the Related Art

In today's modern society, it is common for persons in this country and in numerous foreign countries to eat foods having elevated levels of fat and carbohydrates. It has become widely known, however, that a diet high in fats and carbohydrates is not likely to provide many of the vitamins or other nutrients required to maintain the human body in good general health. Perhaps more importantly, diets high in fats and carbohydrates are more likely to contribute to health problems, such as obesity and/or heart disease, and may result in additional complications for person's with diabetes.

It has been appreciated in recent years that the fat content of foods eaten are a major culprit behind human weight gain. For example, regardless of the type of fat present in a food product, fats have the highest caloric value per gram about 9 calories per gram—of any food group. It is understood that the body tends to store fat for future use, rather than to utilize it immediately, and this factor helps lead to weight-gain. However, in recent years it has been recognized, that there is a connection between the amount of fat stored in the body and the level of cholesterol in the body. A diet high in fats is more likely to result in the development of higher cholesterol levels. As cholesterol has been indicated as a factor in arteriosclerosis or hardening of the arteries, the risk for heart disease and/or a heart attack is elevated when a diet high in fat is followed. Unfortunately, fats also make many food items more tasty—whether butter on bread, dressings on salads, sour cream on potatoes, or frosting on cake—and are therefore, difficult to eliminate entirely from one's diet. Thus, fats usually finds their way into the body, after which a healthy body automatically secretes lipase, an enzyme that accelerates synthesis of fats, i.e., breaking down the fat molecule. The majority of all fats in foods are present in “triglyceride form”, which the body seeks to break down by removing the glycerol molecule from the triglyceride and thereby, release the free fatty acids. Once this occurs, the body is well on its way to absorbing the fat and likely, storing same instead of utilizing it for energy.

It is also generally well understood that when a person eats a food having a high carbohydrate content, such as the sugar in a dessert, ice cream, chocolate bar, etc., a healthy body reacts to the sugar by producing or secreting sufficient levels of insulin as required to properly metabolize the sugars. In particular, the healthy body produces sufficient insulin such that the cells of the body readily absorb glucose molecules where they are stored until they are required to provide the body with energy, at which point the glucose molecules are further converted so that they may provide energy directly to the cells.

Further, it is also generally understood that the glucose that is not directly absorbed into the cells of the body may be converted into a larger polymeric molecule, known as glycogen, in the liver through the process of glycogenesis. Glycogen is not only produced by the liver, but it is stored there for later conversion back into glucose, through the process of glycogenolysis, and the reconverted glucose is then delivered back into the bloodstream for absorption into the cells of the body, as required. In a healthy body, the balance of the glucose/insulin system is carefully controlled by this process of absorption of glucose into the cells, in the presence of insulin, conversion and storage of glucose to glycogen by the liver, and conversion of glycogen back into glucose as the blood glucose level requires replenishment. An additional mechanism, known as gluconeogenesis, allows glucose to be synthesized from amino acids or other non-hexose carbohydrates, when the liver's stores of glycogen are nearly depleted.

Of course, the body of a diabetic reacts differently to the sugars consumed with various carbohydrate foods, by not producing or secreting insulin, or at least in sufficient amounts so as to metabolize the sugars. The excess glucose in the bloodstream of a diabetic has harmful effects, which can be quite serious. However, excess glucose in the bloodstream of even an otherwise healthy person can result in the excess sugar being converted to fat, after which it may no longer be efficiently metabolized by the body. Elevated blood glucose levels have also been attributed to accelerated aging and other health issues in certain individuals. Even the consumption of supposedly healthier foods and drinks high in other complex carbohydrates, such as starches, cellulose, seaweed products, and natural gums, may result in the production of elevated blood glucose levels as the more complex carbohydrates are broken down and converted into simpler sugars, such as glucose.

As can be seen from the foregoing, the longer undigested fats and carbohydrates remain in the body, as often occurs in persons whose digestive processes are not functioning normally, the more likely they will be broken down and/or stored by the body as unwanted fats and sugars. Fortunately, the converse is also true, and the less time food remains in the body, the less likely it is to be broken down and stored as fats and sugars. Of course, the relationship between time and unwanted storage in the body depends on the specific type of fats and carbohydrates consumed by the person, as well as his or her level of physical activity. Thus, among other factors such as the type and amount of foods consumed as well as physical exertion, the maintenance of a relatively normal functioning of the digestive processes, so as to facilitate the elimination of unwanted fats and carbohydrates from the body, is an important factor to be considered with respect to a person's health and well being.

Because of the fact that in this day and age of more sedentary lifestyles, wherein many processed foods are consumed in our everyday diet, and because fats and carbohydrates are an extraordinarily common ingredient in these processed foods, there is a need to inhibit the absorption of fat, enhance the metabolization of carbohydrates, and maintain the normal functioning of the digestive processes, thereby imparting a positive impact on a person's health and well-being. It would thus be beneficial to provide a chemical composition and method to prevent the body from absorbing the fat in such foods once they have been eaten, ideally, by binding fats to each other or to other compounds to limit their absorption by the body. Further, it would be preferable if any such chemical composition and method were developed for it to also enhance the metabolization of carbohydrates so as to reduce the amount of glucose produced, and consequently, the blood glucose level in the human body, thereby minimizing the amount of glucose subsequently stored in the body. In addition, it would be helpful for any such chemical composition and method to aid in the maintenance of the normal functioning of the digestive processes to facilitate elimination of unwanted fats and carbohydrates from the body.

### SUMMARY OF THE INVENTION

The present invention is designed to satisfy the needs in the art and is believed to represent a significant advance in improving a person's health by binding fats, enhancing metabolism of carbohydrates, and maintaining the normal functioning of the digestive processes.

More specifically, the present invention provides a novel, chemical composition for ingestion by humans which is structured to bind to molecules of fat which have been ingested by a person in various types of food. The fat binding components of the inventive chemical composition generally comprise an amount of between 22% and 27% of the total weight, and are structured to bind undigested fats to one another or to other compounds, thereby limiting the absorption of fats by the body. In a preferred embodiment, the fat binding component comprises approximately equal amounts, by weight, of a fibrous agent, such as psyllium, which acts to bind undigested fats, and chitosan, which also acts to bind undigested fats, which together, comprise approximately 25% of the total weight of the chemical composition.

The chemical composition of the present invention is also structured to enhance the metabolism of carbohydrates which have been ingested by a person in various types of food, and more specifically, to limit glucose levels in the bloodstream which may subsequently be stored in the body. The metabolic enhancing components of the inventive composition generally comprise an amount of between 43% and 53% of the total weight of the chemical composition, although in a preferred embodiment, they comprise approximately 47% of the total weight. In one preferred embodiment, the metabolic enhancing components comprise approximately equal amounts by weight of several plant and/or plant extracts. Specifically, one embodiment comprises approximately equal amounts of Garcinia cambogia extract, Gymnema sylvestre, and white kidney bean or Phaseolus vulgaris extract. These components act to limit the reduction of starches to sugars, so as to reduce blood glucose levels and thereby limit the conversion of sugars to fats which may then be stored by the body. In at least one embodiment of the present invention, the metabolic enhancing components further comprise a number of vitamins, minerals, and other beneficial compounds. Specifically, at least one embodiment of the metabolic enhancing components comprises ascorbic acid, better known as Vitamin C, and at least a portion of the Vitamin B-Complex, as well as lesser amounts of choline, inositol, para-aminobenzoic acid, and/or chromium picolinate.

The chemical composition of the present invention is also structured to aid a person's digestion, and as such, the composition preferably includes digestive facilitating components. The digestive facilitating components preferably comprise an amount of generally between approximately 25% and 31% of the total weight of the chemical composition and are structured to maintain the normal functioning of the digestive processes to facilitate the elimination of unwanted fats and carbohydrates from the body. In a preferred embodiment, the digestive facilitating components approximately equal amounts, by weight, of bromelain, papain, and lecithin, in an amount of approximately 28% of the total weight of the chemical composition. In another preferred embodiment, the digestive facilitating components also comprise amounts of peppermint, fructoligosaccharides, and ginger which further help to maintain the normal functioning of the person's digestive processes.

Additionally, a method for using the chemical composition is also described, which comprises the steps of forming a unit dosage tablet or capsule comprising between about 500 mg and 1000 mg of the aforementioned chemical composition in the amounts disclosed above, and having a human ingest at least one but preferably two of these unit dosage capsules with generally about eight ounces of water shortly after having eating a meal. As an alternative, however, the capsules may be consumed prior to eating a meal.

A feature of the chemical composition according to the present invention is that it is moisture activated and, therefore, is inert and can be formed into and stored as conveniently sized capsules or tablets until being ingested by a human and activated by coming into contact with bodily secretions or other liquids.

These and other objects, features and advantages of the present invention will become readily apparent from the detailed description, which follows.

### DETAILED DESCRIPTION OF THE PREFERRED EMBODIMENT

The present invention is directed towards a chemical composition for ingestion by humans which acts to bind fat to limit absorption, enhance metabolization of carbohydrates to reduce blood glucose levels, and to maintain normal functioning of the digestive processes to facilitate the elimination of unwanted fats and carbohydrates from the body. The present invention is also directed to a method for utilizing the inventive chemical composition.

The chemical composition of the present invention preferably comprises a mixture of three distinct groups of components, also termed fractions herein, with each group or fraction being structured to impart a particular desired effect upon ingestion by a person. The three groups or fractions include a fat binding fraction, structured to bind undigested fats in the stomach to limit their absorption by the body, a metabolic enhancer fraction structured to improve the metabolization of carbohydrates, thereby reducing the level of glucose in the bloodstream and subsequent storage in the body, and a digestive aid fraction structured to maintain normal functioning of the digestive processes so as to help eliminate unwanted fats and carbohydrates from the body.

The fat binding fraction of the present invention comprises at least one fibrous agent in an amount of generally between 22% and 27% of the total weight of the chemical composition, and is structured to bind undigested fats to one another or to other compounds, thereby limiting the absorption of fats by the body. In at least one preferred embodiment, the fat binding fraction comprises approximately 25% of the total weight of the chemical composition and may include any of a number of compounds including, but not limited to, psyllium, bran, and in particular, oat bran, the husks of natural oat bran seeds, as well as Plantago ovata seed mucilage. In the more preferred embodiments of the chemical composition, the fibrous agent comprises psyllium, which is a purified fiber product obtained from the dilute acid extract from the seeds of Plantago ovata. Additional embodiments of the chemical composition of the present invention are envisioned which may comprise one or more other fibrous agents which act both as a vehicle for binding undigested fat and as a medium for allowing a human to feel full.

The fat binding fraction of the present invention preferably also comprises chitosan, which also acts to bind undigested fats, thereby further limiting their storage in the body. Chitosan is derived from chitin, which is obtained from the shells of marine crustaceans such as crab, lobster and shrimp, by dissolving the shells' with calcium carbonate, and then removing protein fragments. To obtain chitosan, chitin is exposed to a concentrated sodium hydroxide solution heated to above 135 degrees Celsius, wherein the chitin is deacetylated. Chitosan may be present in the inventive chemical composition in an amount approximately equal to that of the fibrous agent. Although in a most preferred embodiment, chitosan comprises generally an amount of between approximately 10% and 14% of the total weight of the chemical composition, and, ideally, about 12% of the total weight, with the fibrous agent in this embodiment comprising psyllium, also in an amount of generally between approximately 10% and 14% of the total weight of the chemical composition, and ideally, about 12% of the total weight. In this embodiment, the chitosan and the psyllium each comprise an amount of approximately 100 milligrams (mg) per unit dosage of the chemical composition of the present invention. As previously indicated, however, it remains within the scope of the present invention for the chemical composition to comprise one or more other fibrous agents.

The metabolic enhancer fraction of the chemical composition of the present invention generally comprises an amount of between 43% and 53% of the total weight of the chemical composition. In at least one embodiment, the metabolic enhancer fraction comprises approximately 47% of the total weight of the chemical composition. The metabolic enhancer fraction is structured to improve the metabolization of carbohydrates so as to limit the glucose level in the bloodstream, thereby reducing the amount of glucose subsequently stored in the body.

The metabolic enhancer fraction of the present invention comprises a mixture of hydroxycitric acid, also known as Citrimax, which is an active ingredient extracted from the herbal compound Garcinia cambogia, and at least two other major ingredients. Specifically, the metabolic enhancer fraction also comprises, Gymnema sylvestre, and white kidney bean or Phaseolus vulgaris extract, each of which is discussed more fully herein. The Garcinia cambogia extract is an active component that inhibits citrate lyase, an enzyme which is thought to block the conversion of glucose to fat and instead promotes its conversion to glycogen. The inventive chemical composition comprises a predetermined amount of Citrimax or Garcinia cambogia extract of between approximately 10% and 14% of the total weight of the chemical composition, and, more preferably, about 12% of the total weight.

As noted above, the metabolic enhancer fraction of the present invention further comprises Gymnema sylvestre, which is present in an amount of between approximately 10% and 14% of the total weight of the chemical composition, and, ideally, about 12% of the total weight. Gymnema sylvestre is a plant whose saponins (gymnemic acids) are thought to inhibit the intestine's absorption of glucose.

In a preferred embodiment of the present invention, the metabolic enhancer fraction further comprises white kidney bean or Phaseolus vulgaris extract, preferably in an unheated, concentrated form. The Phaseolus vulgaris extract also comprises an amount of between approximately 10% and 14% of the total weight of the chemical composition and, more preferably, about 12% of the total weight. The Phaseolus vulgaris extract is a heat-labile, alpha amylase inhibitor, which is important because alpha amylase is an enzyme secreted by the saliva and the pancreas that breaks down starches to sugars.

In addition to the foregoing, at least one embodiment of the metabolic enhancer fraction of the present invention includes vitamins, minerals, and other beneficial compounds. For example, ascorbic acid, better known as Vitamin C, may be included in powder form comprising an amount of between approximately 5% and 7% of the total weight of the chemical composition, wherein the ascorbic acid serves as an antioxidant for protection against disease and stress. Further, the chemical composition may include at least a portion of the Vitamin B-Complex, comprising, by way of example only, thiamin, riboflavin, niacin, Vitamin B-6, Vitamin B-12, folic acid, and pantothenic acid, which may also be included in powder form. In one preferred embodiment, the Vitamin B-Complex comprises an amount of between approximately 2% and 3% of the total weight of the chemical composition. In at least one embodiment of the chemical composition of the present invention, the metabolic enhancer fraction further comprises choline, which prevents fat deposits in the liver, inositol, and para-aminobenzoic acid, each comprising less than 1% of the total weight of the chemical composition. A further embodiment comprises an amount of chromium picolinate in the metabolic enhancer fraction, in an amount of approximately 0.02% of the total weight of the chemical composition, to increase the binding of insulin to glucose and its internalization into the body's cells to be used as energy, and not converted to fat.

The chemical composition of the present invention also comprises a digestion facilitating components or a digestive aid fraction, as indicated above. The digestive aid fraction generally comprises an amount of between approximately 25% and 31% of the total weight of the chemical composition, and is structured to maintain the normal functioning of the digestive processes to facilitate the elimination of unwanted fats and carbohydrates from the body. In a preferred embodiment, the digestive aid fraction comprises an amount of approximately 28% of the total weight of the chemical composition.

In a preferred embodiment, the digestive aid fraction comprises bromelain, which acts to catalyze the metabolization of proteins, in an amount of between approximately 5% and 7% of the total weight of the chemical composition, and, ideally, in an amount of approximately 6% of the total weight. In at least one embodiment, the digestive aid fraction also comprises papain, a derivative of papaya and also known as vegetable pepsin, which is utilized to treat intestinal disorders. The digestive aid fraction of the present invention comprises papain in an amount of between approximately 5% and 7% and, in one preferred embodiment, in an amount of approximately 6% of the total weight of the chemical composition.

A further embodiment of the digestive aid fraction comprises lecithin, a phospholipid common in plants and animals which is essential for the metabolism of fats. The digestive aid fraction comprises an amount of lecithin of generally between 5% and 7%, however, a preferred embodiment comprises lecithin in an amount of approximately 6% of the total weight of the chemical composition of the present invention.

The digestive aid fraction of the present invention may also comprise peppermint, fructoligosaccharides, and ginger, each of which is believed by the inventor(s) herein to possess properties which help maintain normal functioning of the digestive properties in the body. Each of these components may be present in amounts of between approximately 2% and 4% of the total weight of the chemical composition. In a preferred embodiment, the digestive aid fraction comprises peppermint, fructoligosaccharides, and ginger, each in an amount of approximately 3% of the total weight of the chemical composition.

The inventive chemical composition is preferably formed into either tablets or capsules comprising between 500 mg to 1000 mg, with a preferred embodiment comprising 800 mg of the chemical composition. In one embodiment, each unit dosage tablet or capsule contains at least the following components in the corresponding approximate amounts: 100 mg each of psyllium and chitosan; 100 mg each of Garcinia cambogia, Gymnema sylvestre, and Phaseolus vulgaris extract; 50 mg of Vitamin C; 50 mg each of bromelain, papain, and lecithin; and 25 mg each of peppermint, fructoligosaccharides, and ginger.

In a more preferred embodiment, the inventive chemical composition is formed into unit dosage tablets or capsules each comprising approximately: 100 mg of psyllium; 100 mg of chitosan; 100 mg of Garcinia cambogia; 100 mg of Gymnema sylvestre; 100 mg of Phaseolus vulgaris extract; 50 mg of Vitamin C; 50 mg of bromelain; 50 mg of papain; 50 mg of lecithin; 25 mg of peppermint; 25 mg of fructoligosaccharides; and 25 mg of ginger. In addition, the following vitamins, minerals, and other beneficial compounds will ideally be included in approximately the following amounts: 19.75 mg of Vitamin B-Complex comprising 1.50 mg of thiamin, 1.50 mg of riboflavin, 1.50 mg of niacin, 5 mg of Vitamin B-6, 0.05 mg of Vitamin B-12, 0.20 mg of folic acid, and 10 mg of pantothenic acid; 5 mg of choline; 5 mg of inositol; and 5 mg of para-aminobenzoic acid, and/or excipients. Further, a preferred dosage for this embodiment is two tablets shortly after both lunch and dinner.

It will be appreciated that a capsule containing between about 500 mg and 1000 mg has a size and overall dimension which is readily suited for being comfortably swallowed by a person, the capsule could be formed to contain more or less of the chemical composition (with ratios of the ingredients of the composition similar to that disclosed herein) and thereby be somewhat larger or smaller, and still function in the intended manner when ingested by a person.

The method of the present invention comprises the steps of forming a tablet or capsule of a unit dosage comprising about 800 mg of the chemical composition disclosed herein and having the human ingest at least one unit dosage capsule with generally about eight ounces of water shortly after he or she has eaten a meal, such as generally about fifteen to twenty minutes afterwards. Ideally, the human will ingest one or two of the unit dosage capsules after a meal, but may ingest up to about three capsules if the meal eaten is especially large and/or has a particularly high content of fats and/or carbohydrates. Upon being ingested by a human, each capsule begins to disintegrate and releases or otherwise facilitates activation of the chemical composition contained therein, typically in about thirty (30) minutes, and often in less time. As an alternative, the human may ingest the one or two unit dosage capsules shortly before a meal. In addition, the human may be directed to ingest generally about eight ounces of water upon waking up in the morning, and ideally, there is an additional step of having the human ingest generally about eight ounces of water between meals.

Since many modifications, variations and changes in detail can be made to the described preferred embodiment of the invention, it is intended that all matters in the foregoing description be interpreted as illustrative and not in a limiting sense. As one example, while the inventive chemical composition disclosed herein has been referred to as useful if consumed by and implemented with humans, it is contemplated that other animals may treated and derive benefits from the chemical composition, and their treatment should also be considered within the scope and spirit of the present invention. Thus, the scope of the invention should be determined by the appended claims and their legal equivalents.

Now that the invention has been described,

1. A chemical composition for ingestion by a human which binds fat, enhances metabolism, and aids digestion, said composition comprising:
a predetermined amount of a fat binding fraction;
a predetermined amount of a metabolic enhancer fraction; and
a predetermined amount of a digestive aid fraction.

2. The chemical composition as recited in claim 1 wherein said predetermined amount of said fat binding fraction is between approximately 22% and 27% by weight of said composition; said predetermined amount of said metabolic enhancer fraction is between approximately 43% and 53% by weight of said composition; and said predetermined amount of said digestive aid fraction is between approximately 25% and 31% by weight of said composition.

3. A chemical composition as recited in claim 2 wherein said fat binding fraction comprises approximately equal amounts of chitosan and psyllium, by weight.

4. A chemical composition as recited in claim 2 wherein said metabolic enhancer fraction comprises approximately equal amounts of Garcinia cambogia extract, Gymnema sylvestre, and Phaseolus vulgaris extract, by weight.

5. A chemical composition as recited in claim 4 wherein said metabolic enhancer fraction further comprises an amount of Vitamin C equal to approximately 50% of said amount of said Garcinia cambogia extract, by weight.

6. A chemical composition as recited in claim 5 wherein said metabolic enhancer fraction further comprises an amount of Vitamin B-Complex equal to approximately 20% of said amount of said Garcinia cambogia extract, by weight.

7. A chemical composition as recited in claim 2 wherein said digestive aid fraction comprises approximately equal amounts of bromelain, papain, and lecithin, by weight.

8. A chemical composition as recited in claim 7 wherein said digestive aid fraction further comprises equal amounts of peppermint, fructoligosaccharides, and ginger, each in an amount equal to approximately 50% of said amount of said bromelain, by weight.

9. A chemical composition as recited in claim 2 wherein said predetermined amount of said fat binding fraction comprises approximately 25% of said composition, said predetermined amount of said metabolic enhancer fraction comprises approximately 47% of said composition, and said predetermined amount of said digestive aid fraction comprises approximately 28% of said composition, by weight.

10. A chemical composition as recited in claim 1 wherein said chemical composition is formed into a plurality of unit dosage capsules each comprising between about 180 and 220 mg of said fat binding fraction, between about 350 and 425 mg of said metabolic enhancer fraction, and between about 200 and 250 mg of said digestive aid fraction.

11. A chemical composition as recited in claim 10 wherein said fat binding fraction comprises approximately 100 mg each of chitosan and psyllium.

12. A chemical composition as recited in claim 10 wherein said metabolic enhancer fraction comprises approximately 100 mg each of Garcinia cambogia extract, Gymnema sylvestre, and Phaseolus vulgaris extract.

13. A chemical composition as recited in claim 12 wherein said metabolic enhancer fraction further comprises approximately 50 mg of Vitamin C.

14. A chemical composition as recited in claim 13 wherein said metabolic enhancer fraction further comprises approximately 20 mg of Vitamin B-Complex.

15. A chemical composition as recited in claim 14 wherein said Vitamin B-Complex comprises approximately 1.5 mg of thiamin, 1.5 mg of riboflavin, 1.5 mg of niacin, 5 mg of Vitamin B-6, 0.05 mg of Vitamin B-12, 0.20 mg of folic acid, and 10 mg of pantothenic acid.

16. A chemical composition as recited in claim 14 wherein said metabolic enhancer fraction further comprises approximately 5 mg each of choline, inositol, para-aminobenzoic acid.

17. A chemical composition as recited in claim 16 wherein said metabolic enhancer fraction further comprises approximately 0.20 mg of chromium picolinate.

18. A chemical composition as recited in claim 10 wherein said digestive aid fraction comprises approximately 50 mg each of bromelain, papain, and lecithin.

19. A chemical composition as recited in claim 18 wherein said digestive aid fraction further comprises approximately 25 mg each of peppermint, fructoligosaccharides, and ginger.

20. A method of binding fat, enhancing metabolism, and aiding digestion in a human, said method comprising the steps of:
forming a capsule comprising a chemical composition including approximately 100 mg of chitosan, 100 mg of psyllium, 100 mg of Garcinia cambogia extract, 100 mg of Gymnema Sylvestre, 100 mg of Phaseolus vulgaris extract, 50 mg of Vitamin C, 50 mg of bromelain, 50 mg of papain, 50 mg of lecithin, 25 mg of peppermint, 25 mg of fructoligosaccharides, 25 mg of ginger, and 20 mg of Vitamin B-Complex; and
having the human ingest at least one capsule with generally about eight ounces of water generally about fifteen minutes after consuming a meal.
